(19) United States
(12) Patent Application Publication
(10) Pub. No.: US 2019/0247106 A1
(43) Pub. Date: Aug. 15, 2019

(54) BALLOON INFLATION RATE CONTROLLER FOR CRYOGENIC BALLOON CATHETER SYSTEM

(71) Applicant: Cryterion Medical, Inc., Carlsbad, CA (US)

(72) Inventor: Chadi Harmouche, Saint-Laurent (CA)

(21) Appl. No.: 16/275,897

(22) Filed: Feb. 14, 2019

Related U.S. Application Data
(60) Provisional application No. 62/631,033, filed on Feb. 15, 2018.

(51) Int. Cl. A61B 18/02; A61M 25/10

(52) U.S. Cl. CPC A61B 18/02; A61B 2018/00351; A61M 25/10184

(57) ABSTRACT

A cryogenic balloon catheter system for use by an operator in treating a condition in a patient comprises a fluid source that selectively retains a fluid, and a catheter including a first balloon configured to receive the fluid from the fluid source. A balloon inflation rate controller is configured to control an inflation rate of the first balloon.

### CROSS-REFERENCE TO RELATED APPLICATION

This application claims priority to Provisional Application No. 62/631,033, filed Feb. 15, 2018, which is herein incorporated by reference in its entirety.

### TECHNICAL FIELD

The present disclosure relates to medical devices and methods for treating cardiac arrhythmias. More specifically, the disclosure relates to devices and methods for cardiac cryoablation.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few. In particular, catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. The energy delivery component of the system is typically at or near the most distal (i.e. farthest from the user or operator) portion of the catheter, and often at the tip of the catheter.

Various forms of energy can be used to ablate diseased heart tissue. These can include radio frequency (RF), cryogenics, ultrasound and laser energy, to name a few. During a cryoablation procedure, with the aid of a guide wire, the distal tip of the catheter is positioned adjacent to targeted cardiac tissue, at which time energy is delivered to create tissue necrosis, rendering the ablated tissue incapable of conducting electrical signals. The dose of the energy delivered is a critical factor in increasing the likelihood that the treated tissue is permanently incapable of conduction. At the same time, delicate collateral tissue, such as the esophagus, the bronchus, and the phrenic nerve surrounding the ablation zone can be damaged and can lead to undesired complications. Thus, the operator must finely balance delivering therapeutic levels of energy to achieve intended tissue necrosis while avoiding excessive energy leading to collateral tissue injury.

Atrial fibrillation (AF) is one of the most common arrhythmias treated using catheter ablation. In the earliest stages of the disease, paroxysmal AF, the treatment strategy involves isolating the pulmonary veins from the left atrial chamber. Recently, the use of techniques known as “balloon cryotherapy” catheter procedures to treat AF have increased. In part, this stems from the balloon cryotherapy's ease of use, shorter procedure times and improved patient outcomes.

During balloon cryoablation procedures, the operator needs to ensure proper balloon-tissue contact and vein occlusion before starting an ablation to increase probability of vein isolation. More particularly, complete occlusion of each pulmonary vein with the cryoballoon is required for adequate antral ablation and electrical isolation. Without pulmonary vein occlusion, blood flow over the balloon during ablation decreases the likelihood of sufficient lesion formation. Thus, at the outset of a balloon cryoablation procedure, the operator endeavors to position and inflate one or more balloons of the balloon catheter to achieve the desired balloon-tissue contact and vein occlusion. Traditionally, the operator will inflate the balloon with no cooling fluid provided therein, position the balloon at the ostium of the vein, and verify occlusion using methods such as injection contrast and fluoroscopy. Unfortunately, such inflation methods have not always proved satisfactory.

### SUMMARY

In Example 1, a cryogenic balloon catheter system for use by an operator in treating a condition in a patient, the cryogenic balloon catheter system comprising a fluid source, a catheter and a balloon inflation rate controller. The fluid source selectively retains a fluid, and the catheter includes a first balloon configured to receive the fluid from the fluid source. The balloon inflation rate controller is configured to control an inflation rate of the first balloon.

In Example 2, the cryogenic balloon catheter system of Example 1, wherein the balloon inflation rate controller is configured to control a flow rate at which the fluid is provided to the first balloon such that the inflation rate is between a maximum inflation rate and a minimum inflation rate.

In Example 3, the cryogenic balloon catheter system of either of Examples 1 or 2, wherein the balloon inflation rate controller includes a rate control selector that enables the operator to select a desired inflation rate of the first balloon among a plurality of discrete inflation rates between the maximum inflation rate and the minimum inflation rate.

In Example 4, the cryogenic balloon catheter system of either of Examples 1 or 2, wherein the balloon inflation rate controller includes a rate control selector that enables the operator to select a desired inflation rate of the first balloon anywhere along a continuum between the maximum inflation rate and the minimum inflation rate.

In Example 5, the cryogenic balloon catheter system of any of Examples 1-4, wherein the balloon inflation rate controller includes at least one of (i) an inflation pressure controller that is configured to control an inflation pressure of the fluid that is provided to the first balloon; (ii) a fluid delivery timer that is configured to control a length of time that the fluid is provided to the first balloon; or (iii) a fluid delivery direction controller that is configured to control a direction by which the fluid is provided to the first balloon.

In Example 6, the cryogenic balloon catheter system of any of Examples 1-5, wherein the balloon inflation rate controller includes each of (i) the inflation pressure controller that is configured to control an inflation pressure of the fluid that is provided to the first balloon; (ii) the fluid delivery timer that is configured to control a length of time that the fluid is provided to the first balloon; and (iii) the fluid delivery direction controller that is configured to control a direction by which the fluid is provided to the first balloon.

In Example 7, the cryogenic balloon catheter system of any of Examples 1-6, further comprising a fluid injection line that is coupled in fluid communication to and extends between the fluid source and the first balloon, and a fluid return line that is coupled in fluid communication to and extends between the fluid source and the first balloon; and wherein the fluid delivery direction controller controls the fluid to be provided to the first balloon via one or both of the fluid injection line and the fluid return line.

In Example 8, the cryogenic balloon catheter system of Example 7, wherein the fluid injection line is a first size and the fluid return line is a second size that is smaller than the first size.

In Example 9, the cryogenic balloon catheter system of any of Examples 1-8, further comprising a second balloon that is positioned to substantially encircle the first balloon.

In Example 10, the cryogenic balloon catheter system of any of Examples 1-9, wherein the fluid from the fluid source is liquid nitrous oxide or liquid nitrogen.

In Example 11, the cryogenic balloon catheter system of any of Examples 1-10, further comprising a graphical display that is configured to display information to the operator; wherein the balloon inflation rate controller is accessible to the operator via the graphical display.

In Example 12, a method for controlling an inflation rate of a balloon usable in a cryogenic balloon catheter system for treating a condition in a patient, the method comprising the steps of receiving a fluid from a fluid source within the balloon of the cryogenic balloon catheter system, and controlling an inflation rate of the first balloon via an inflation rate controller.

In Example 13, the method of Example 12, wherein controlling the inflation rate includes selecting, via a rate control selector, a desired inflation rate of the first balloon from among a plurality of inflation rates between a maximum inflation rate and a minimum inflation rate.

In Example 14, the method of either of Examples 12 or 13, wherein controlling the inflation rate includes at least one of (i) controlling an inflation pressure of the fluid that is provided to the first balloon with an inflation pressure controller; (ii) controlling a length of time that the fluid is provided to the first balloon with a fluid delivery timer; or (iii) controlling a direction by which the fluid is provided to the first balloon with a fluid delivery direction controller.

In Example 15, the method of any of Examples 12-14, wherein the cryogenic balloon catheter system comprises a fluid injection line that is coupled in fluid communication to and extends between the fluid source and the first balloon, and a fluid return line that is coupled in fluid communication to and extends between the fluid source and the first balloon, and wherein controlling the direction by which the fluid is provided to the first balloon includes selectively controlling the fluid to be provided to the first balloon via one or both of the fluid injection line and the fluid return line.

In Example 16, a cryogenic balloon catheter system for use by an operator in treating a condition in a patient, the cryogenic balloon catheter system comprising a balloon catheter, a control console, a fluid injection line and a fluid return line. The balloon catheter includes an expandable balloon defining a cryochamber, and the control console includes a fluid source and a balloon inflation rate controller. The fluid injection line is in fluid communication with the expandable balloon and the fluid source, and the fluid return line is in fluid communication with the expandable balloon and the fluid source. The balloon inflation rate controller is configured to control an inflation rate of the expandable balloon by controlling a rate of delivery of fluid from the fluid source to the expandable balloon via one or both of the fluid injection line and the fluid return line.

In Example 17, the cryogenic balloon catheter system of Example 16, wherein the balloon inflation rate controller is configured to control a flow rate at which the fluid is provided to the expandable balloon such that the inflation rate is between a maximum inflation rate and a minimum inflation rate.

In Example 18, the cryogenic balloon catheter system of Example 17, wherein the balloon inflation rate controller includes a rate control selector that enables the operator to select the inflation rate.

In Example 19, the cryogenic balloon catheter system of any of Examples 16-18, wherein the balloon inflation rate controller includes at least one of (i) an inflation pressure controller that is configured to control an inflation pressure of the fluid that is provided to the expandable balloon; (ii) a fluid delivery timer that is configured to control a length of time that the fluid is provided to the expandable balloon; or (iii) a fluid delivery direction controller that is configured to control a direction by which the fluid is provided to the expandable balloon.

In Example 20, the cryogenic balloon catheter system of any of Examples 16-19, wherein the control console further includes a graphical display, and wherein the rate control selector is operable by the operator via the graphical display.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a simplified schematic side view illustration of a patient and one embodiment of a cryogenic balloon catheter system including a balloon inflation rate controller having features of the present disclosure; and

FIG. 2 is a simplified schematic view illustration of a portion of the patient and a portion of an embodiment of the cryogenic balloon catheter system that includes the balloon inflation rate controller.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure are described herein in the context of a balloon inflation rate controller for use within a cryogenic balloon catheter system. In particular, the balloon inflation rate controller is configured to enable the operator to effectively control the rate of balloon inflation to provide the best opportunity to achieve the desired balloon-tissue contact and vein occlusion, while still providing overall time efficiency for the cryoablation procedure.

Those of ordinary skill in the art will realize that the following detailed description of the present disclosure is illustrative only and is not intended to be in any way limiting. Other embodiments of the present disclosure will readily suggest themselves to such skilled persons having the benefit of this disclosure. Reference will now be made in detail to implementations of the present disclosure as illustrated in the accompanying drawings.

In the interest of clarity, not all of the routine features of the implementations described herein are shown and described. It will, of course, be appreciated that in the development of any such actual implementation, numerous implementation-specific decisions must be made to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound and laser energy, as non-exclusive examples. The present disclosure is intended to be effective with any or all of these and other forms of energy.

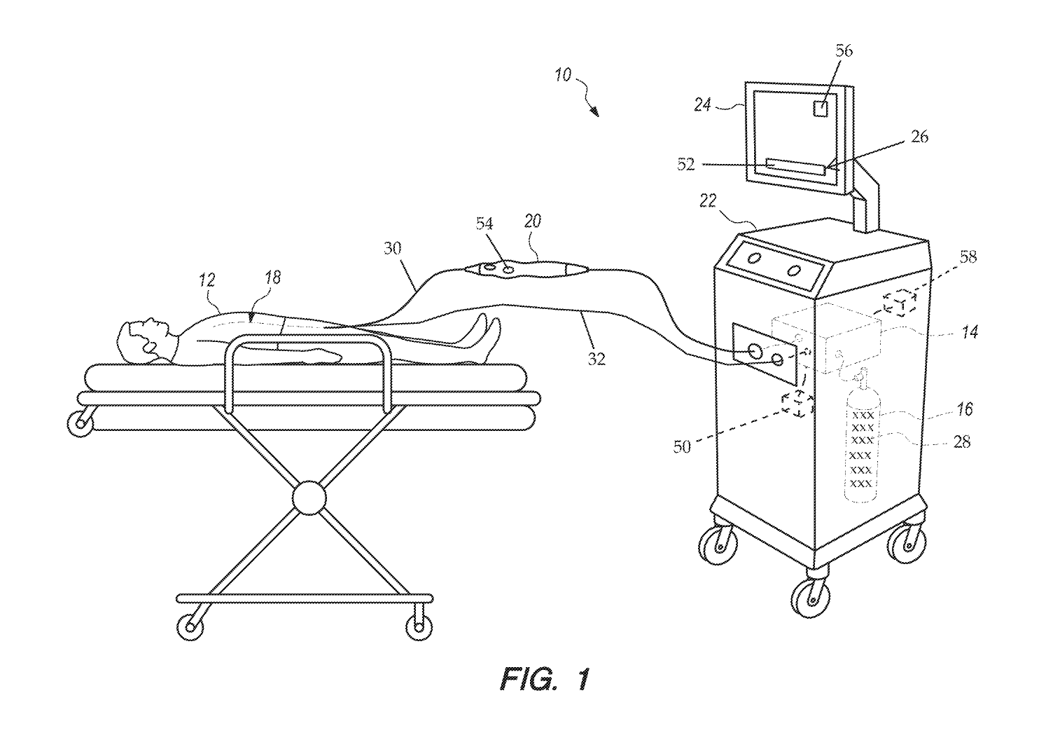

FIG. 1 is a simplified schematic side view illustration of an embodiment of a medical device 10 for use with a patient 12, which can be a human being or an animal. Although the specific medical device 10 illustrated and described herein pertains to and refers to a cryogenic balloon catheter system 10, it is understood and appreciated that other types of medical devices 10 or systems can equally benefit by the teachings provided herein. For example, in certain non-exclusive alternative embodiments, the present disclosure can be equally applicable for use with any suitable types of ablation systems and/or any suitable types of catheter systems. Thus, the specific reference herein to use as part of a cryogenic balloon catheter system is not intended to be limiting in any manner.

The design of the cryogenic balloon catheter system 10 can be varied. In certain embodiments, such as the embodiment illustrated in FIG. 1, the cryogenic balloon catheter system 10 can include one or more of a control system 14 (illustrated in phantom), a fluid source 16 (illustrated in phantom), a balloon catheter 18, a handle assembly 20, a control console 22, a graphical display 24, and a balloon inflation rate controller 26 (sometimes referred to herein as an “inflation rate controller”).

It is understood that although FIG. 1 illustrates the structures of the cryogenic balloon catheter system 10 in a particular position, sequence and/or order, these structures can be located in any suitably different position, sequence and/or order than that illustrated in FIG. 1. It is also understood that the cryogenic balloon catheter system 10 can include fewer or additional components than those specifically illustrated and described herein.

In various embodiments, the control system 14 is configured to monitor and control various processes of the ablation procedures performed with the cryogenic balloon catheter system 10. More specifically, the control system 14 can monitor and control release and/or retrieval of a cooling fluid 28 (e.g., a cryogenic fluid) to the balloon catheter 18, e.g., via a fluid injection line 30, and/or from the balloon catheter 18, e.g., via a fluid return line 32. The control system 14 can also control various structures that are responsible for maintaining and/or adjusting a flow rate and/or pressure of the cryogenic fluid 28 that is released to the balloon catheter 18 during the cryoablation procedure. In such embodiments, the cryogenic balloon catheter system 10 delivers ablative energy in the form of cryogenic fluid 28 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18.

Further, or in the alternative, the control system 14 can receive data and/or other information (hereinafter sometimes referred to as “sensor output”) from various structures within the cryogenic balloon catheter system 10, and/or can receive data and/or other information (hereinafter sometimes referred to as “inflation rate control output”) from the inflation rate controller 26. In some embodiments, the control system 14 can receive, monitor, assimilate and/or integrate the sensor output, the inflation rate control output, and/or any other data or information received from any structure within the cryogenic balloon catheter system 10 in order to control the operation of the balloon catheter 18. As provided herein, in various embodiments, the control system 14 can initiate and/or terminate the flow of cryogenic fluid 28 to the balloon catheter 18 based on the sensor output and/or the inflation rate control output.

Still further, or in the alternative, the control system 14 can control positioning of portions of the balloon catheter 18 within the body of the patient 12, and/or can control any other suitable functions of the balloon catheter 18.

As shown in FIG. 1, in certain embodiments, the control system 14 can be positioned substantially within the control console 22. Alternatively, at least a portion of the control system 14 can be positioned in one or more other locations within the cryogenic balloon catheter system 10, e.g., within the handle assembly 20.

The fluid source 16 contains the cryogenic fluid 28, which is delivered to and from the balloon catheter 18 with or without input from the control system 14 during a cryoablation procedure. Once the ablation procedure has initiated, the cryogenic fluid 28 can be delivered and the resulting gas, after a phase change, can be retrieved from the balloon catheter 18, and can either be vented or otherwise discarded as exhaust. Additionally, the type of cryogenic fluid 28 that is used during the cryoablation procedure can vary. In one non-exclusive embodiment, the cryogenic fluid 28 can include liquid nitrous oxide. However, any other suitable cryogenic fluid 28 can be used. For example, in one non-exclusive alternative embodiment, the cryogenic fluid 28 can include liquid nitrogen.

The design of the balloon catheter 18 can be varied to suit the specific design requirements of the cryogenic balloon catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a healthcare professional (also referred to herein as an “operator”). As used herein, a healthcare professional and/or an operator can include a physician, a physician's assistant, a nurse and/or any other suitable person and/or individual. In certain embodiments, the balloon catheter 18 is positioned within the body of the patient 12 utilizing at least a portion of the sensor output that is received by the control system 14. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue (not shown). While specific reference is made herein to the balloon catheter 18, as noted above, it is understood that any suitable type of medical device and/or catheter may be used.

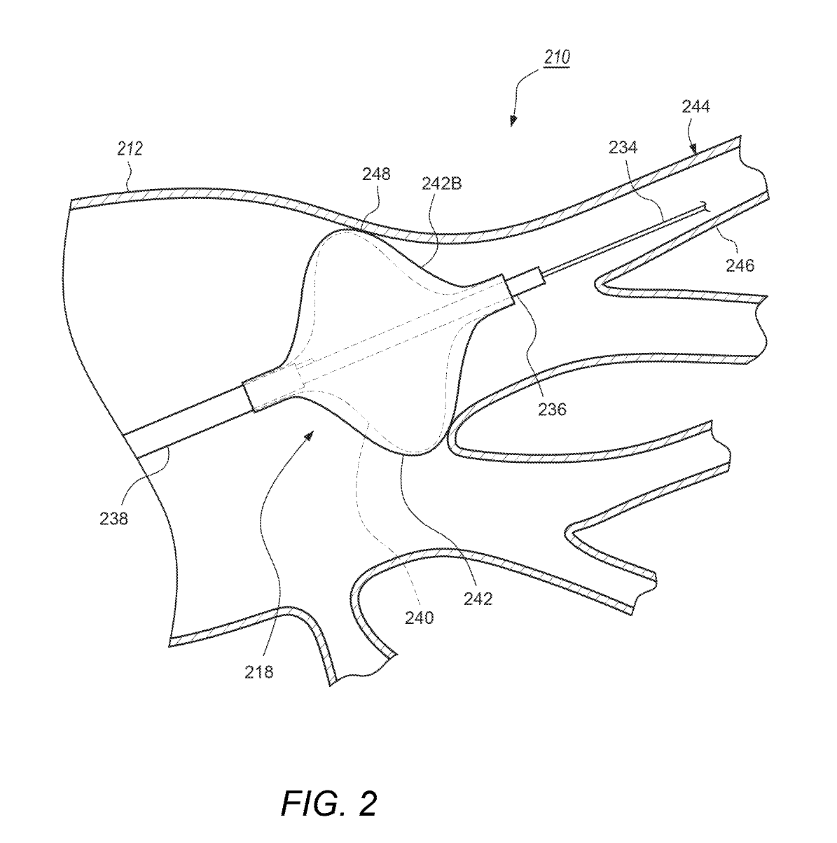

Additional features and aspects of embodiments of the balloon catheter 18 and the positioning of the balloon catheter 18 are illustrated in greater detail in FIG. 2. In particular, FIG. 2 is a simplified schematic view illustration of a portion of one embodiment of the cryogenic balloon catheter system 210, i.e. a portion of the balloon catheter 218, and a portion of a patient 212.

In balloon cryotherapy systems, it is common that two balloons are used to create a cryo-chamber near the distal tip of the balloon catheter 218. More specifically, in the embodiment illustrated in FIG. 2, the balloon catheter 218 includes one or more of a guidewire 234, a guidewire lumen 236, a catheter shaft 238, an inner balloon 240 and an outer balloon 242. The balloons 240, 242 are configured such that the inner balloon 240 receives the cryogenic fluid 28 (illustrated in FIG. 1), and the outer balloon 242 surrounds the inner balloon 240. The outer balloon 242 acts as part of a safety system to capture the cryogenic fluid 28 in the event of a leak from the inner balloon 240. It is understood that the balloon catheter 218 can include other structures as well. However, for the sake of clarity, these other structures have been omitted from the Figures. Additionally, it is further appreciated that in some alternative embodiments, the balloon catheter 18 includes only a single balloon.

In the embodiment illustrated in FIG. 2, the balloon catheter 218 is positioned within the circulatory system 244 of the patient 212. The guidewire 234 and guidewire lumen 236 are inserted into a pulmonary vein 246 of the patient 212, and the catheter shaft 238 and the balloons 240, 242 are moved along the guidewire 234 and/or the guidewire lumen 236 to be positioned near an ostium 248 of the pulmonary vein 246.

During use, the inner balloon 240 can be partially or fully inflated so that at least a portion of the inner balloon 240 expands against at least a portion of the outer balloon 242. Once the inner balloon 240 is sufficiently inflated, an outer surface 242B of the outer balloon 242 can then be positioned within the circulatory system 244 of the patient 212 to abut and/or substantially form a seal with the ostium 248 of the pulmonary vein 246 to be treated.

The inner balloon 240 and the outer balloon 242 can be formed from any suitable materials. For example, in some embodiments, the inner balloon 240 can be formed from a sturdy material to better inhibit leaks of the cryogenic fluid 28 that is received therein, and the outer balloon 242 can be made from a relatively compliant material to ensure better contact and positioning between the outer balloon 242 and the pulmonary vein 246.

As noted above, during balloon cryoablation procedures, the operator needs to ensure proper balloon-tissue contact and vein occlusion before starting an ablation to increase probability of vein isolation. To do so, the operator will inflate at least one of the balloons 240,242, e.g., the inner balloon 240, position the balloons 240, 242 at the ostium 248 of the pulmonary vein 246, and verify occlusion using methods such as injection contrast and fluoroscopy. The rate at which the balloons 240, 242 inflate may have critical effects on the success of the occlusion, as well as having an impact on the overall procedure duration. For example, a fast inflation rate may, under certain conditions, cause the balloons 240, 242 to push back from the pulmonary vein 246 and prevent good occlusion. Conversely, slow inflation rate may reduce procedure risks if inflated in the pulmonary vein 246, and may also reduce undesired location by allowing the operator time to react if improper location is detected. Thus, it can be desired to provide balloon inflation that is slow enough to inhibit the disadvantages noted for fast inflation procedures, while still providing certain desired time efficiencies for the overall cryoablation procedure.

In various embodiments, as provided in detail herein, the rate of balloon inflation can be controlled by the operator via the inflation rate controller 26 (illustrated in FIG. 1). Details of various embodiments of the inflation rate controller 26 will be provided herein below.

Returning now back to FIG. 1, in various embodiments, the handle assembly 20 is handled and used by the operator to operate, position and control the balloon catheter 18. The design and specific features of the handle assembly 20 can vary to suit the design requirements of the cryogenic balloon catheter system 10. In the embodiment illustrated in FIG. 1, the handle assembly 20 is separate from, but in electrical and/or fluid communication with the control system 14, the fluid source 16, the graphical display 24, and the inflation rate controller 26. In some embodiments, the handle assembly 20 can integrate and/or include at least a portion of the control system 14 within an interior of the handle assembly 20. It is understood that the handle assembly 20 can include fewer or additional components than those specifically illustrated and described herein. Additionally, in certain embodiments, the handle assembly 20 can include circuitry (not shown in FIG. 1) that can include at least a portion of the control system 14. Alternatively, the circuitry can transmit electrical signals such as the sensor output and/or the inflation rate control output, or otherwise provide data to the control system 14 as described herein. Further, or in the alternative, the circuitry can receive electrical signals or data from the inflation rate controller 26. In one embodiment, the circuitry can include a printed circuit board having one or more integrated circuits, or any other suitable circuitry.

Still further, in certain embodiments, the handle assembly 20 can be used by the operator to initiate and/or terminate the cryoablation process, e.g., to start the flow of the cryogenic fluid 28 to the balloon catheter 18 in order to ablate certain targeted heart tissue of the patient 12.

In the embodiment illustrated in FIG. 1, the control console 22 includes at least a portion of the control system 14, the fluid source 16, the graphical display 24, and the inflation rate controller 26. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the graphical display 24.

During cryoablation procedures, the balloon catheter 18 and the control console 22 must be mechanically connected to allow the flow of cryogenic fluid 28 from the control console 22 to the balloon catheter 18 and back to the control console 22. Generally, the cryogenic fluid 28 flows in a liquid phase to the balloon catheter 18, e.g., to the inner balloon 240 (illustrated in FIG. 2) of the balloon catheter 18. The cryogenic fluid 28 then undergoes a phase change and returns to the control console 22 as exhaust in a gaseous phase with the assistance of a vacuum pump 50.

In various embodiments, the graphical display 24 is electrically connected to the control system 14 and the inflation rate controller 26. Additionally, the graphical display 24 provides the operator of the cryogenic balloon catheter system 10 with information that can be used before, during and after the cryoablation procedure. For example, the graphical display 24 can provide the operator with information based on the sensor output, the inflation rate control output, and any other relevant information that can be used before, during and after the cryoablation procedure. The specifics of the graphical display 24 can vary depending upon the design requirements of the cryogenic balloon catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time, e.g., during an ablation procedure. Further, in various embodiments, the graphical display 24 can include one or more colors, different sizes, varying brightness, etc., that may act as alerts to the operator. Additionally, or in the alternative, the graphical display 24 can provide audio data or information to the operator.

As provided herein, the inflation rate controller 26 is configured to enable the operator to effectively control the rate of balloon inflation to provide the best opportunity to achieve the desired balloon-tissue contact and vein occlusion, while still providing overall time efficiency for the cryoablation procedure. The inflation rate controller 26 can be positioned in any suitable manner within the cryogenic balloon catheter system 10. For example, as illustrated in FIG. 1, in certain embodiments, at least a portion of the inflation rate controller 26 can be positioned within the control console 22 and/or adjacent to the control system 14. Additionally, or in the alternative, in some embodiments, at least a portion of the inflation rate controller 26 can be positioned within and/or substantially adjacent to the handle assembly 20. Further, or in the alternative, at least a portion of the inflation rate controller 26 can be accessible via the graphical display 24. Still further, or in the alternative, the inflation rate controller 26 can be positioned in another suitable manner at any suitable location(s) within the cryogenic balloon catheter system 10.

Additionally, the design of the inflation rate controller 26 can be varied to suit the requirements of the cryogenic balloon catheter system 10 and/or to suit the specifications of the operator. In various embodiments, as shown in FIG. 1, the inflation rate controller 26 can include one or more of a rate control selector 52, an inflation pressure controller 54, a fluid delivery timer 56, and a fluid delivery direction controller 58. Further, or in the alternative, the inflation rate controller 26 can include additional components or fewer components than those specifically illustrated in FIG. 1, and/or the components of the inflation rate controller 26 can be positioned in a different manner than what is specifically shown in FIG. 1.

The rate control selector 52 enables the operator to select a desired rate for the inflation of the balloons 240, 242 of the balloon catheter 18. In certain embodiments, the rate control selector 52 can be accessible to the operator via the graphical display 24. Alternatively, in other embodiments, the rate control selector 52 can be accessible within the handle assembly 20. Still alternatively, the rate control selector 52 can be accessible via another portion of the cryogenic balloon catheter system 10.

Additionally, the rate control selector 52 can be provided in any suitable form, and can enable the operator to select from any desired number of rate settings. For example, in certain embodiments, the rate control selector 52 can be provided in the form of a selection button, which can provide options of fast inflation (maximum inflation rate), slow inflation (minimum inflation rate), and any suitable number of inflation rate options between fast inflation and slow inflation. Alternatively, the rate control selector 52 can be provided in the form of a slider or a dial that enable the operator to make discrete rate selections between a maximum inflation rate and a minimum inflation rate. Still alternatively, the inflation rate options can be provided along a continuum, thus enabling any desired inflation rate options between a maximum inflation rate and a minimum inflation rate. Yet alternatively, the rate control selector 52 can be provided in another suitable form.

In addition to the various rate settings that can be controlled and/or selected via the rate control selector 52, the actual mechanisms for controlling the balloon inflation rate within the cryogenic balloon catheter system 10 can be provided in any suitable manner. For example, in the embodiment illustrated in FIG. 1, the mechanisms for controlling the balloon inflation rate can be provided in the form of one or more of the inflation pressure controller 54, the fluid delivery timer 56, and the fluid delivery direction controller 58. Alternatively, the mechanisms for controlling the balloon inflation rate can be provided in another suitable form.

The inflation pressure controller 54 is configured to control the balloon inflation rate by controlling the inflation pressure of the fluid that is being used to inflate one or more of the balloons 240, 242, e.g., the inner balloon 240. In particular, by controlling the inflation pressure of the fluid that is being used to inflate the balloons 240, 242, the inflation pressure controller 54 is effectively controlling the actual flow rate of the fluid to the balloons 240, 242. For example, a higher inflation pressure will result in a higher flow rate of fluid to the balloons 240, 242; and thus provide an inflation rate closer to the maximum inflation rate. Conversely, a lower inflation pressure will result in a lower flow rate of fluid to the balloons 240, 242, and thus provide an inflation rate closer to the minimum inflation rate.

In the embodiment shown in FIG. 1, the inflation pressure controller 54 is provided within the handle assembly 20. Alternatively, the inflation pressure controller 54 can be provided within the control console 22 and/or adjacent to the control system 14, and/or the inflation pressure controller 54 can be provided in or near another component of the cryogenic balloon catheter system 10.

In certain embodiments, the fluid delivery timer 56 is configured to control the duration of delivery of fluid, e.g., the cryogenic fluid 28, to the balloons 240, 242 during the inflation of the balloons 240, 242. Stated in another manner, in such embodiments, the fluid delivery timer 56 controls the length of time that the fluid, e.g., the cryogenic fluid 28, is delivered to the balloons 240, 242 during the balloon inflation period of the cryoablation procedure.

In some embodiments, the fluid delivery timer 56 can be coupled to and/or provided within the graphical display 24. Alternatively, the fluid delivery timer 56 can be provided within the handle assembly 20, the control console 22, the control system 14, and/or within another suitable component of the cryogenic balloon catheter system 10.

Additionally, in certain embodiments, the fluid delivery direction controller 58 is configured to control the direction by which fluid, e.g., the cryogenic fluid 28, is directed to the balloons 240, 242 during the balloon inflation period of the cryoablation procedure. For example, based on the chosen inflation rate setting, the fluid delivery direction controller 58 can control the cryogenic fluid 28 to be directed to the balloons 240, 242 of the balloon catheter 18 via the fluid injection line 30, via the fluid return line 32, or via both the fluid injection line 30 and the fluid return line 32. In some embodiments, the fluid injection line 30 can be somewhat larger than the fluid return line 32, thus enabling faster fluid flow through the fluid injection line 30 than the fluid return line 32. In such embodiments, the fluid delivery direction controller 58 can control the fluid to be directed to the balloons 240, 242 via the fluid injection line 30 during the balloon inflation period when a somewhat faster inflation rate is desired. Conversely, the fluid delivery direction controller 58 can control the fluid to be directed to the balloons 240, 242 via the fluid return line 32 during the balloon inflation period when a somewhat slower inflation rate is desired. Further, with such design, it is appreciated that a maximum inflation rate is attainable when the fluid delivery direction controller 58 controls the fluid to be directed to the balloons 240, 242 via both the fluid injection line 30 and the fluid return line 32 during the balloon inflation period.

It is further understood that the use of one or more of the inflation pressure controller 54, the fluid delivery timer 56 and the fluid delivery direction controller 58 can be combined in any suitable manner to provide even greater variability to the inflation rate options provided by the inflation rate controller 26.

It is understood that although a number of different embodiments of the cryogenic balloon catheter system 10 and the balloon inflation rate controller 26 have been illustrated and described herein, one or more features of any one embodiment can be combined with one or more features of one or more of the other embodiments, provided that such combination satisfies the intent of the present disclosure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A cryogenic balloon catheter system for use by an operator in treating a condition in a patient, the cryogenic balloon catheter system comprising:
a fluid source that selectively retains a fluid;
a catheter including a first balloon configured to receive the fluid from the fluid source; and
a balloon inflation rate controller configured to control an inflation rate of the first balloon.

2. The cryogenic balloon catheter system of claim 1, wherein the balloon inflation rate controller is configured to control a flow rate at which the fluid is provided to the first balloon such that the inflation rate is between a maximum inflation rate and a minimum inflation rate.

3. The cryogenic balloon catheter system of claim 2, wherein the balloon inflation rate controller includes a rate control selector that enables the operator to select a desired inflation rate of the first balloon among a plurality of discrete inflation rates between the maximum inflation rate and the minimum inflation rate.

4. The cryogenic balloon catheter system of claim 2, wherein the balloon inflation rate controller includes a rate control selector that enables the operator to select a desired inflation rate of the first balloon anywhere along a continuum between the maximum inflation rate and the minimum inflation rate.

5. The cryogenic balloon catheter system of claim 1, wherein the balloon inflation rate controller includes at least one of (i) an inflation pressure controller that is configured to control an inflation pressure of the fluid that is provided to the first balloon; (ii) a fluid delivery timer that is configured to control a length of time that the fluid is provided to the first balloon; or (iii) a fluid delivery direction controller that is configured to control a direction by which the fluid is provided to the first balloon.

6. The cryogenic balloon catheter system of claim 5, wherein the balloon inflation rate controller includes each of (i) the inflation pressure controller that is configured to control an inflation pressure of the fluid that is provided to the first balloon; (ii) the fluid delivery timer that is configured to control a length of time that the fluid is provided to the first balloon; and (iii) the fluid delivery direction controller that is configured to control a direction by which the fluid is provided to the first balloon.

7. The cryogenic balloon catheter system of claim 5, further comprising a fluid injection line that is coupled in fluid communication to and extends between the fluid source and the first balloon, and a fluid return line that is coupled in fluid communication to and extends between the fluid source and the first balloon; and wherein the fluid delivery direction controller controls the fluid to be provided to the first balloon via one or both of the fluid injection line and the fluid return line.

8. The cryogenic balloon catheter system of claim 7, wherein the fluid injection line is a first size and the fluid return line is a second size that is smaller than the first size.

9. The cryogenic balloon catheter system of claim 1, further comprising a second balloon that is positioned to substantially encircle the first balloon.

10. The cryogenic balloon catheter system of claim 1, wherein the fluid from the fluid source is liquid nitrous oxide or liquid nitrogen.

11. The cryogenic balloon catheter system of claim 1, further comprising a graphical display that is configured to display information to the operator; wherein the balloon inflation rate controller is accessible to the operator via the graphical display.

12. A method for controlling an inflation rate of a balloon usable in a cryogenic balloon catheter system for treating a condition in a patient, the method comprising:
receiving a fluid from a fluid source within the balloon of the cryogenic balloon catheter system; and
controlling an inflation rate of the first balloon via an inflation rate controller.

13. The method of claim 12, wherein controlling the inflation rate includes selecting, via a rate control selector, a desired inflation rate of the first balloon from among a plurality of inflation rates between a maximum inflation rate and a minimum inflation rate.

14. The method of claim 13, wherein controlling the inflation rate includes at least one of (i) controlling an inflation pressure of the fluid that is provided to the first balloon with an inflation pressure controller; (ii) controlling a length of time that the fluid is provided to the first balloon with a fluid delivery timer; or (iii) controlling a direction by which the fluid is provided to the first balloon with a fluid delivery direction controller.

15. The method of claim 14, wherein the cryogenic balloon catheter system comprises a fluid injection line that is coupled in fluid communication to and extends between the fluid source and the first balloon, and a fluid return line that is coupled in fluid communication to and extends between the fluid source and the first balloon, and wherein controlling the direction by which the fluid is provided to the first balloon includes selectively controlling the fluid to be provided to the first balloon via one or both of the fluid injection line and the fluid return line.

16. A cryogenic balloon catheter system for use by an operator in treating a condition in a patient, the cryogenic balloon catheter system comprising:
a balloon catheter including an expandable balloon defining a cryochamber;
a control console including a fluid source and a balloon inflation rate controller;
a fluid injection line in fluid communication with the expandable balloon and the fluid source; and
a fluid return line in fluid communication with the expandable balloon and the fluid source,
wherein the balloon inflation rate controller is configured to control an inflation rate of the expandable balloon by controlling a rate of delivery of fluid from the fluid source to the expandable balloon via one or both of the fluid injection line and the fluid return line.

17. The cryogenic balloon catheter system of claim 16, wherein the balloon inflation rate controller is configured to control a flow rate at which the fluid is provided to the expandable balloon such that the inflation rate is between a maximum inflation rate and a minimum inflation rate.

18. The cryogenic balloon catheter system of claim 17, wherein the balloon inflation rate controller includes a rate control selector that enables the operator to select the inflation rate.

19. The cryogenic balloon catheter system of claim 18, wherein the balloon inflation rate controller includes at least one of (i) an inflation pressure controller that is configured to control an inflation pressure of the fluid that is provided to the expandable balloon; (ii) a fluid delivery timer that is configured to control a length of time that the fluid is provided to the expandable balloon; or (iii) a fluid delivery direction controller that is configured to control a direction by which the fluid is provided to the expandable balloon.

20. The cryogenic balloon catheter system of claim 19, wherein the control console further includes a graphical display, and wherein the rate control selector is operable by the operator via the graphical display.
